(19) United States
(12) Patent Application Publication
(10) Pub. No.: US 2004/0167540 A1
(43) Pub. Date: Aug. 26, 2004

(54) Marking instrument

(76) Inventor: Georg Gerten, Bonn (DE)

Correspondence Address: Fay Sharpe Fagan Minnich & McKee, 7th Floor, 1100 Superior Avenue, Cleveland, OH, 44114-2518, US

(21) Appl. No.: 10/478,083

(22) PCT Filed: May 15, 2002
(86) PCT No.: PCT/EP02/05349

(30) Foreign Application Priority Data
May 18, 2001 (DE) 101 24 708.7

(51) Int. Cl. A61B 17/00

(52) U.S. Cl. 606/116

(57) ABSTRACT

Marking instrument for producing marks, in particular for uses in optical surgery, having a head (12) comprising marking elements (14) and adjustable in relation to the object (16) to be marked, and an instrument handle (11) arranged thereon. So as to be able for the marks to be produced in exactly the desired angular position relative to the normal to the earth's surface, the marking instrument is provided with a positioning device (18; 20) displaying and/or adjusting the angular position of the marking elements according to the invention, whereby the positioning device can be a level or a verticality indicator or an adjustment mechanism, which provides automatically for the right alignment of the instrument head

The invention relates to a marking instrument for producing in particular horizontal and/or vertical marks for surgery, in particular optical surgery uses, with an instrument head comprising marking elements, adjustable in relation to the object to be marked, and an instrument handle arranged thereon.

Before surgical interventions, it is often required to apply surgical marks on the future surgical area while the patient is standing or sitting, so that the surgeon has got important reference points during surgery which generally takes place while the patient is lying, so as to be able to undertake the surgical intervention necessary for surgery at the right place and with the right orientation. Such marks are for example important in plastic surgery. They are particularly important with surgical interventions at the eye, where it is often important, during insertion of intra-ocular lenses, during cornea surgery or refractive surgery, to show the surgeon the correct horizontal or vertical axis of the eye.

Up to now, such marks are applied to the object to be marked by means of a marking instrument by hand, in that the marking elements of the marking instrument used are at first provided with marking ink similar to a die, and are then pressed against the surface to be marked, for example the cornea of an eye to be operated on. For this, the patient mainly stands or sits upright, whereby he has to hold his head straight during the marking of the cornea of the eye and has to locate an object at eye level arranged at a certain distance. The surgeon then aligns the marking instrument according to visual judgement in such a manner that the marking elements take up the desired position, that is are in particular horizontal and/or vertical, before he presses the instrument head against the area to be marked, on which the marking elements which were previously provided with marking ink leave the desired mark.

The alignment of the marking instrument just by hand according to visual judgement regularly results in that the exactness during marking leaves a lot to be desired, and thereby a for example vertical or horizontal axis at the eye before cornea surgery interventions can never be defined with a certainty of 100%, but always depends on the visual judgement of the person who applies the mark.

It is the object of the invention to avoid this disadvantage and to create a marking instrument which makes it possible to apply marks on a surgery area in exactly the desired, preferably horizontal and/or vertical alignment.

This object is solved by means of the invention by a positioning device displaying and/or adjusting the desired angular position of the marking elements relative to the normal of the earth. The positioning device can thereby preferably comprise a level or vertical indicator, with the help of which the person applying the mark can recognise exactly when the marking instrument is in the position relative to the surgery field, in which the marking elements can take up the desired angular position relative to the normal of the earth, that is, are aligned in particular exactly horizontally and/or vertically.

The level or verticality indicator is preferably arranged on the instrument handle. It can comprise a display which is conveniently arranged in the vicinity of the instrument head, so that the person applying the mark can at the same time keep an eye on the display of the level or verticality indicator and the instrument head with the marking elements mounted thereon, while he/she moves the marking instrument against the surgery field to be marked.

In a preferred embodiment of the invention, the level or verticality indicator can in a simple manner essentially consist of a sight glass filled with liquid apart from a gas bubble, as is used with spirit levels. Liquid and gas in the sight glass are conveniently chosen in such a manner that the sight glass is not destroyed during a hot steam sterilisation which is usually used for surgical instruments.

A particularly good exactness during the alignment of the marking instrument can be reached when the level or verticality indicator essentially consists of an electronic verticality encoder and an electric or electronic display preferably arranged in the region of the instrument head, for example a small light emitting diode which lights up when the marking instrument is held exactly in the position in which the marking elements take up the desired angular position relative to the normal of the earth. The verticality encoder and the power source necessary for its operation can for example be arranged in the instrument handle.

A preferred embodiment of the invention results when the positioning device comprises an adjustment mechanism loaded by gravity, adjusting the instrument head relative to the instrument handle, which can for example have a verticality weight effective at the instrument head and a pivot bearing, around which the instrument head can turn relative to the instrument handle. With this embodiment of the invention, the instrument head adjusts itself automatically into the desired angular position relative to the normal of the earth, that is, independent of the angular position in which the instrument head is held. The person applying the mark at the surgery field only has to keep the instrument head still until the verticality weight has been dragged into the lowest possible position by the attraction force of the earth and has thereby aligned the instrument head in the correct position. The pivot bearing has preferably a dampening element assigned thereto which prevents that the instrument head only reaches its statically stable position after oscillating to and fro several times, in which the verticality weight is in its lowest possible position.

Further characteristics and advantages of the invention result from the following description and the drawing, in which preferred embodiments of the invention are described in more detail by means of examples. It shows:

FIG. 1 a first embodiment of a marking instrument according to the invention for applying marks on the cornea of an eye in one view;

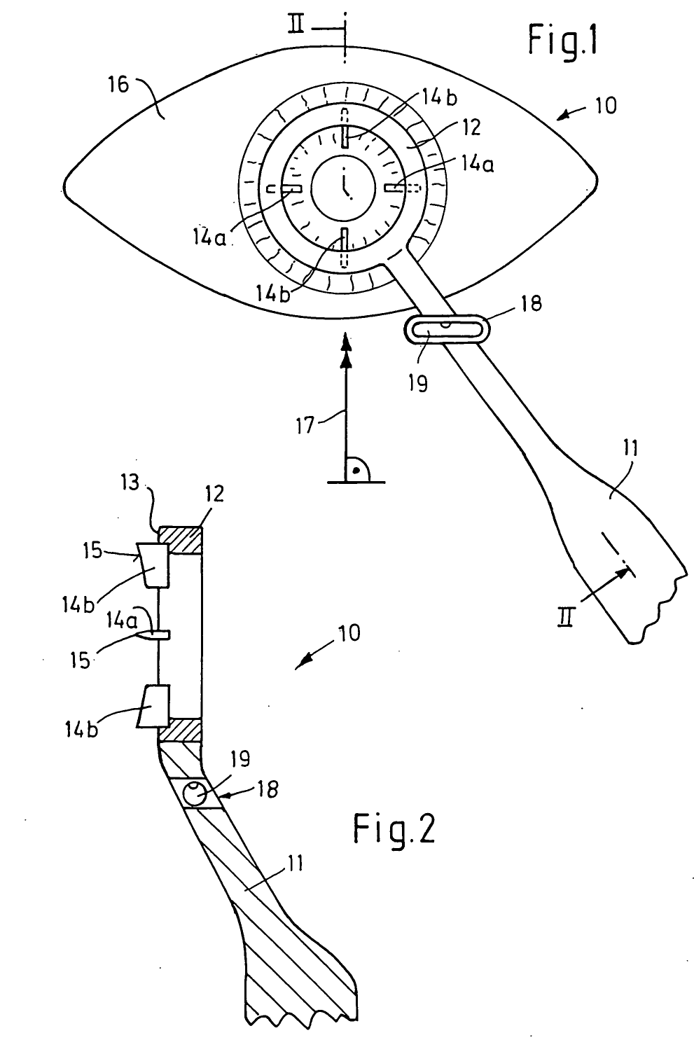

FIG. 2 the object of FIG. 1 in section along line II-II;

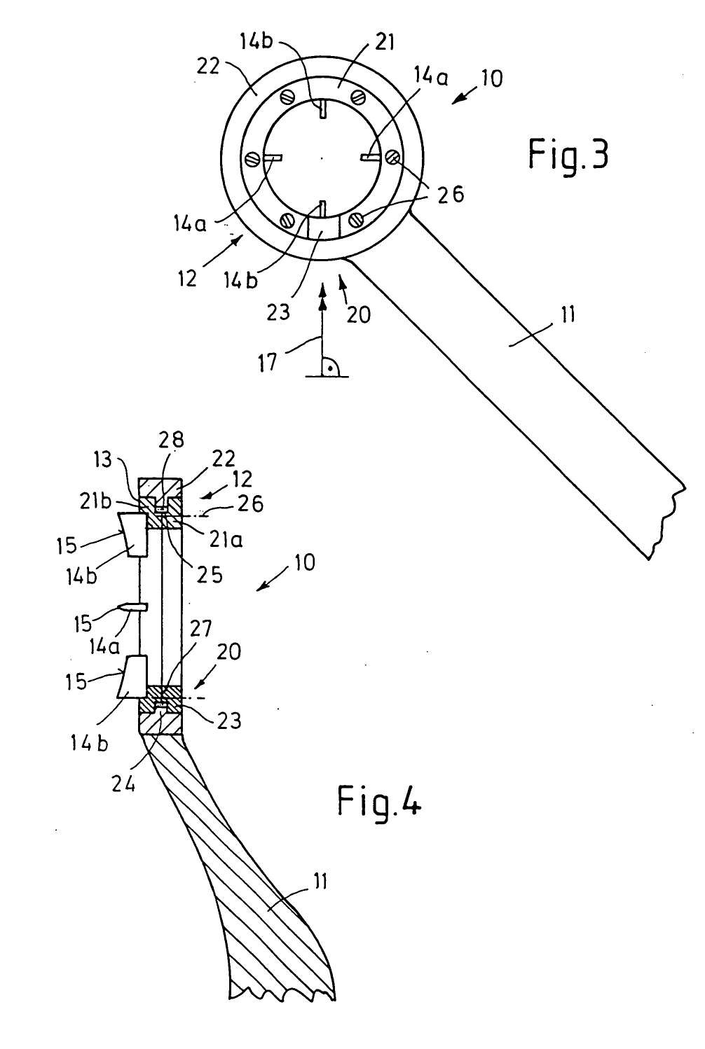

FIG. 3 a second embodiment of the marking instrument in one view; and

FIG. 4 the object of FIG. 3 in a section along line IV-IV.

The marking instruments 10 shown in the drawings serve to draw out marks on the cornea of a patient who will be subjected to optical surgery, which marks show the surgeon the horizontal and/or vertical axial position of the eye. The marks are, similar to a die print, transferred to the cornea of the eye with the help of the marking instrument 10, while the patient is standing or sitting upright, whereby he has to hold the head straight and has to locate a previously defined vanishing point with the eye to be marked, while the person undertaking the marking aligns the marking instrument in the correct position and arranges it against the cornea of the patient.

As results from the drawings, the marking instrument essentially consists of an instrument handle 11 and an instrument head 12 arranged thereon which comprises altogether four marking elements 14 which are arranged with the same angular distance from one another at the front side 13 facing the eye to be marked, which elements are adapted to the contour of the eye to be marked at their free front side. The marking elements form die surfaces with their front edges 15, which surfaces can be dyed with a suitable die ink or the like, which will be transferred to the cornea of the patient if the marking elements come into contact with the eye of the patient. With the examples of the embodiments shown, marks for the horizontal and the vertical axial position shall be applied to the cornea of the eye 16 to be operated on; accordingly, the instrument head is provided with two horizontal marking elements 14a and two vertical marking elements 14b, which shall be aligned exactly vertical or parallel to the normal of the earth during the marking process, so as to exactly define the horizontal or vertical axis on the cornea of the patient. For this, the marking instrument 10 according to the invention is provided with a positioning device displaying or adjusting the desired angular position of the marking elements relative to the normal 17 of the earth, which enables the person who applies the marking to do this with a great exactness.

In the first embodiment shown in FIGS. 1 and 2, the positioning device consists essentially of a level device 18 with a sight glass 19 filled with liquid apart from a gas bubble, which glass is arranged on the instrument handle 11 in the vicinity of the instrument head 12 and which shows the user, similar to a spirit level, in which position of the instrument handle 11 the instrument head with the marking elements fixedly secured thereon will take up the correct position so as to produce an exact horizontal or vertical mark on the surface to be marked, that is, the cornea of the patient. For this, the person applying the marking examines the display in the sight glass 19 and holds the instrument handle 11 of the marking instrument at such an angle at which the gas bubble in the sight glass 19 is exactly between two adjustment lines applied thereon. The marking instrument is only then mounted on the surface to be marked, without again changing the angular position of the instrument handle relative to the normal of the earth. In this manner, it is possible to apply the marks with their desired angle relative to the normal of the earth with a great exactness.

In the embodiment shown in FIGS. 3 and 4, the positioning device comprises an adjustment mechanism 20 adjusting the instrument head relative to the instrument handle, which automatically provides for a correct alignment of the marking elements before the application of the marks on the cornea of the eye. For this, the instrument head is formed as a highly precisely manufactured swivel ring 21, which is freely pivoted in a reception hub 22 arranged at the front end of the instrument handle. The swivel ring which is otherwise made from a light material is provided with a vertical weight 23 in a small ring segment running coaxially to the two vertical marking elements 14b, which weight is always drawn downward by the attraction force of the earth in the direction of the normal of the earth during the use of the marking instrument. That is, the vertical weight provides that the vertical marking instruments 14b are aligned in a parallel manner and that the horizontal marking elements 14a are aligned in a right-angled manner to the normal 17 of the earth, in that the swivel ring 21 in the reception hub 22 adjusts itself into this desired position due to the attraction force of the earth which draws the vertical weight down. It is thereby immaterial at which angle the instrument head 11 is held by the person who shall apply the mark on the surface to be marked.

It can be seen from FIG. 4 that the swivel ring 21 is secured against falling out of the reception hub 22. For this, the reception hub comprises a circumferential annular web 24, which engages a circumferential radial groove 25 at the swivel ring. For the mounting of the swivel ring, it is manufactured in two parts, whereby the two parts 21a, 21b are inserted from both sides of the reception hub 22 into it and are connected to one another in a detachable manner by means of several connecting screws 26. As a material for the swivel ring and the reception hub are considered in particular materials having a very low thermal expansion coefficient, for example ceramic materials, so that the free rotatability of the swivel ring in the hub is not influenced by fluctuations of the ambient temperature. So as to avoid a strong oscillation of the swivel ring in the reception hub, the gap 27 formed between the web and the radial groove 25 can be filled with a dampening liquid 28, which then provides that the vertical weight 23 does not or at least not notably oscillate to the opposite side during its way in the shown, lowest position.

The invention is not restricted to the shown and described examples of embodiments, but various changes and additions are feasible without leaving the scope of the invention. The level or verticality indicator used does not have to be a mere optical-mechanical instrument described in the first embodiment, but an electronic verticality indicator can be used which displays the correct alignment of the instrument with the help of an electric or electronic display as for example a light emitting diode or the like. It is of course also possible to provide a gravity-loaded adjustment mechanism as well as a level or verticality indicator, whereby the level or verticality indicator is then obviously not arranged on the instrument handle but on the instrument head of the marking instrument, that is the part, the correct alignment of which is important during the application of the marks. Suitable changes of the marking instrument are of course also feasible, which can then be used in plastic or cosmetic surgery so as to apply marks on the surgery field for the surgeon.

1. Marking instrument for producing of in particular horizontal and/or vertical marks for surgery uses, in particular optical surgery uses, having an instrument head (12) comprising marking elements (14), adjustable against the object (16) to be marked, and an instrument handle (11) arranged thereon, and with a positioning device (18; 20) adjusting the desired angular position of the marking elements (14) relative to the normal (17) of the earth, whereby the positioning device comprises an gravity-loaded adjustment mechanism (20) adjusting the instrument head (12) relative to the instrument handle (11).

2. Marking instrument according to claim 1, characterised in that the adjustment mechanism (20) comprises a verticality weight (23) effective at the instrument head (21), as well as a pivot bearing (21, 22) around which the instrument head (12) can turn relative to the instrument handle (11).

3. Marking instrument according to claim 2, characterised by a dampening element (28) assigned to the pivot bearing (21, 22).

4. Marking instrument according to one of claims 1 to 3, characterised in that the positioning device comprises a level or verticality indicator (18).

5. Marking instrument according to claim 4, characterised in that the level or verticality indicator (18) is arranged on the instrument head (11)

6. Marking instrument according to claim 4 or 5, characterised in that the level or verticality indicator (18) comprises a display which is arranged in the vicinity of the instrument head (11).

7. Marking instrument according to one of claims 4 to 6, characterised in that the level or verticality indicator (18) essentially consists of a sight glass (18) filled with a liquid apart from a gas bubble.

8. Marking instrument according to one of claims 4 to 6, characterised in that the level or verticality indicator (18) essentially consists of an electronic verticality encoder and an electric or electronic display arranged preferably in the region of the instrument head (12).
